(19) United States
(12) Patent Application Publication
(10) Pub. No.: US 2004/0038875 A1
(43) Pub. Date: Feb. 26, 2004

(54) Alpha 7nicotinic receptor screening assays

(76) Inventors: Susan Adele Greenfield, Oxford (GB); Martin Westwell, Oxford (GB)

Correspondence Address: Nixon & Vanderhye, 8th Floor, 1100 North Glebe Road, Arlington, VA, 22201-4714, US

(21) Appl. No.: 10/240,096

(22) PCT Filed: Mar. 29, 2001
(86) PCT No.: PCT/GB01/01401

(51) Int. Cl. A61K 38/17; G01N 33/53; G01N 33/567

(52) U.S. Cl. 514/12; 435/7.2

(57) ABSTRACT

The present invention relates to screening assays for functional analogues and antagonists of a polypeptide fragment of acetylcholinesterase (AChE), which is believed to exhibit activity corresponding to non-enzymatic function of AchE in the brain. These assays stem from linkage of such non-enzymatic activity with a target site on the homomeric alpha 7 nicotinic receptor. Antagonists thus identified which are capable of formulation for passage through the blood-brain barrier may be advantageous therapeutic agents for the treatment of a number of neurodegenerative diseases, in particular Alzheimer's Disease, Parkinson's Disease and Motor Neuron Disease.

The present invention relates to screening assays for selecting agonists and antagonists for non-enzymatic biological activity of acetylcholinesterase (AChE), which is believed to be mediated in the brain by a polypeptide fragment of the enzyme. In particular, it relates to such assays stemming from identifying the receptor site for a 14 mer fragment of AChE (SEQ. ID. No. 1) as a modulatory site present on the homomeric alpha 7 nicotinic receptor. Antagonists thus identified which are capable of formulation for passage through the blood-brain barrier are envisaged as therapeutic agents for the treatment of a number of neurodegenerative diseases, in particular, for example, Alzheimer's Disease, Parkinson's Disease and Motor Neuron Disease.

### BACKGROUND TO THE INVENTION

It has for some years been proposed that AChE has a non-enzymatic action in the brain, which appears to share close parallels with the action of amyloid precursor protein (APP). Evidence has previously been presented indicating that this non-enzymatic action of AChE underlies a trophic function in developing brains but if activated in adult brains leads to neurodegenerative disorders. This hypothesis for neurodegenerative disease causation is currently of particular interest in relation to, for example, Alzheimer's Disease, Parkinson's Disease and Motor Neuron Disease.

Although Alzheimer's Disease and Parkinson's Disease have different clinical profiles, it has long been acknowledged that the underlying pathologies can overlap. Causation of both diseases can be attributed to different degrees of disruption to. neuronal groups within the globally-projecting neurons extending from the spinal cord to midbrain (referred to as the “isodendritic core”; Rosser, British Medical Journal, (1981) 283, 1588-1598). Studies of this group of neurons have revealed 30 important differences from the rest of the brain (Woolf, Neuroscience (1996) 74, 625-651). Importantly, they have been found to retain the capacity in adult brain for not only axonal regeneration, but also for proliferation. It has previously been suggested that vulnerability of global neurons to degeneration might be associated with aberrant activation of a developmental mechanism in response to local insult. More recently, evidence has been presented leading to linkage of this hypothesis to proposed trophic function of AChE in developing brains mediated by promotion of Ca²⁺ entry into immature neurons (Greenfield, Spring Research News (1997) 2-3; Greenfield, Brit. Med. J. (1998) 317, 19-26; Webb et al., Eur. J. Neurosci.(1996) 8, 837-841; Holmes et al., J. Neuro. Res. (1997) 49, 1-12.). Significantly, although sub-populations of global neurons contain different neurotransmitters, irrespective of whether they are cholinergic neurons acted upon by acetylcholine, they contain AChE (Greenfield, Neurochem. Int.,1996). Furthermore, it is recognised that immature neurons can withstand higher levels of intracellular calcium than their mature counterparts (Eimerl and Schramm, J. Neurochem. (1994) 62, 1223-1226). Indeed, it has been shown that an amount of calcium that will be beneficial in developing neurons will kill their mature counterparts.

The global neuronal population of cells contain not only neurons associated with Alzheimer's Disease and Parkinson's Disease, but also neurons associated with Motor Neuron Disease. Motor neurons have been shown to release AChE (Rodriguez et al., J. Neurol. Sci. (1997) 152, Suppl. 1: S54-61) and embryonic spinal rat motor neurons have also been reported to be sensitive to its trophic action in culture (Kreutzberg et al., Advances in Neurol. (1974) 12, 269-281; Brimjoin, Prog: Neurobiol. (1983) 21, 291-322; Moreno et al., Brain Research (1996) 718, 13-24; Bataillé et al., Eur. J. Neurosci. (1998) 10 (2) 560-572). Hence, there is reason to believe that Alzheimer's Disease, Parkinson's Disease and Motor Neuron Disease have a single underlying causality related to non-enzymic function of AChE.

Published International Application WO 97/35962 discloses a 14 mer biologically active fragment of AChE (Synaptica Peptide) having the sequence AEFHRWSSYMVHWK (SEQ. ID. No. 1), which corresponds to amino acid residues 535 to 548 of the mature protein. Evidence is presented in the same International Application that Synaptica Peptide represents a portion of AChE retaining its capacity to mediate non-enzymatic biological effects. An in vivo counterpart is known to be cleaved from AChE, but the exact nature of the polypeptide fragment which is ultimately responsible in the brain for mediating non-enzymatic activity of AChE remains to be elucidated.

As shown in FIG. 1 of WO 97/35962, and FIG. 1 of the present specification, the sequence of Synaptica Peptide is conserved between AChE of different species, including human and rat AChE, and exhibits similarity to a region of human APP (the region at the N-terminus of the Aβ1-42 fragment which has been associated with Alzheimer's Disease). However, the same sequence or a closely similar sequence has not been found in any butyrylcholinesterase, which like AChE hydrolyses acetylcholine. As also reported in WO 97/35962, electrophysiological studies with slices of adult guinea pig midbrain showed that Synaptica Peptide initially enhances calcium potentials in neurons of the substantia nigra induced either by N-methyl-D-aspartate (NMDA) or by direct depolarisation of neurons. With sustained application of Synaptica Peptide for a few minutes, however, a reduction in calcium entry is observed. This is in keeping with so much calcium entering the neurons that calcium channels are shut-off. The same modulation of calcium influx is not observed with a comparable peptide fragment from butyrylcholinesterase or Aβ1-42.

It has since been shown that Synaptica Peptide can also enhance Ca²⁺ flux into hippocampal neurons which eventually switches off calcium channels. This is of particular interest in relation to Alzheimer's Disease since the hippocampus is the major site of degeneration neuropathology associated with that disease.

On the basis of further studies with Synaptica Peptide, and a biotinylated and amidated version of the same peptide, the target receptor site for Synaptica Peptide is now proposed to be an allosteric modulatory site present on the homomeric alpha 7 nicotinic receptor. This receptor has previously been identified as a nicotinic receptor having high calcium permeability, which is blocked by α-bungarotoxin. By transfecting Xenopus oocytes with mRNA for rat alpha 7 subunits, functional homomeric alpha 7 nicotinic receptors having 5 alpha 7 subunits have previously been obtained for study in vitro (Séquéla et al., J. Neurosci. (1993) 13, 596-604). Published International Application WO 94/20617 also describes cloning of the cDNA for the human alpha 7 nicotinic receptor subunit and engineering of human cells and Xenopus oocytes to express functional homomeric alpha 7 nicotinic receptors. It is believed that such receptors mimic homomeric alpha 7 nicotinic receptors in vivo (Chen and Patrick, J. Biol. Chem. (1997) 272, 24024-24029; Drisdel and Green, J. Neurosci.(2000) 20, 133-139). Such receptors appear transiently in developing brain and occur in regions where AChE is believed to have non-enzymatic function (Broide et al., Neurosci. (1995) 67, 83-94; Broide et al., J. Neurosci. (1996) 16, 2956-2971; Kim et al., Develop. Brain Res. (1995) 85, 283-287; Tengelsen et al., Brain Res. (1992) 594, 10-18). Alpha 7-subunit containing nicotinic receptors, which are sensitive to α-bungarotoxin, have also been found in adrenomedullary chromaffin cells (Lopez et al., Proc. Natl. Acad. Sci. USA (1998) 95, 14184-14189; Criado et al., J. Neurosci. (1997) 117, 6554-6664). Importantly, it has also been reported that alpha 7 subunit mRNA can be found in post-mortem brain tissue from the hippocampus of Alzheimer's Disease patients (Hellström-Lindhal et al., Mol. Brain Res. (1999) 66, 94-103).

That alpha 7 nicotinic receptors are subject to allosteric modulation via binding of ligands at a site distinct from the site for binding of ion-channel agonists has previously also been shown. For example, ivermectin, in the μM range, has been found to strongly enhance acetyl choline-evoked currents through neuronal chick and human homomeric alpha 7 nicotinic receptors in oocytes (Krause et al., Mol. Pharm. (1998) 53, 283-294). The allosteric site targeted by ivermectin in such studies is believed to be distinct from the binding site on alpha 7 nicotinic receptors for Aβ1-42. Binding of Aβ1-42to such receptors has been reported, for example, in Published International Application no. WO 99/62505, but, as indicated above, Aβ1-42 has not been found to have a modulatory effect on induced Ca²⁺ flux. Previously it has been proposed that potentiators capable of acting at the ivermectin site to increase Ca²+flux could be of interest to compensate for the deleterious effects of neurodegenerative disorders. The additional linkage of non-enzymatic function of AChE with an allosteric modulatory site on brain alpha 7 nicotinic receptors importantly now provides for the first time impetus for identification of antagonists of modulatory ligands on such receptors, particularly Synaptica Peptide, in relation to devising new treatments for Alzheimer's Disease and other neurological disorders aimed at preventing, or at least inhibiting, neuronal deterioration.

### SUMMARY OF THE INVENTION

The finding that Synaptica Peptide targets a modulatory site on brain alpha 7 nicotinic receptors provides the foundation for new assays for screening for both functional analogues and antagonists of that peptide.

Thus, in one aspect, the present invention provides use of an alpha 7 nicotinic receptor, preferably a human alpha 7 nicotinic receptor, or a functional analogue thereof, to determine whether a compound is capable of acting as a functional analogue or antagonist of the polypeptide of SEQ. ID. No. 1 on said receptor. Where a native alpha 7 nicotinic receptor is employed in its normal membrane environment, it will be identified by means of inhibition by an alpha 7 nicotinic receptor blocker such as α-bungarotoxin. By functional analogue in this context will be understood a variant of a native alpha 7 nicotinic receptor which retains a modulatory binding site for Synaptica Peptide and ability to exhibit induced Ca²⁺ permeability which is influenced by binding of Synaptica Peptide to the same variant.

In one embodiment, there is thus now provided a method for determining the ability of a compound to act as an antagonist of the polypeptide of SEQ. ID. No. 1 (Synaptica Peptide), which comprises determining whether said compound can inhibit binding of Synaptica Peptide or a functional analogue thereof to its target site on an alpha 7 nicotinic receptor or functional analogue of said receptor and thereby antagonise the modulatory effect of Synaptica Peptide or its analogue on induced ion flux, e.g. Ca²⁺ flux, through the receptor. It will be appreciated that such a method may be applied to screen compounds as part of a screening programme aimed at identifying compounds for use in preventing, inhibiting or reversing neurological disorders believed to be associated with non-enzymatic function of AChE, especially Alzheimer's Disease, Parkinson's Disease and Motor Neuron Disease.

In such a method, Synaptica Peptide may be employed or any compound which is capable of mimicking the modulatory effect of Synaptica peptide at its modulatory binding site on an alpha 7 nicotinic receptor. It will be appreciated that such functional analogues of Synaptica Peptide include,. but are not limited to, variants of Synaptica Peptide having one or more additions and/or deletions and/or substitutions, e.g. conservative substitutions compared to SEQ. ID. No. 1 which result in retention of its calcium channel modulatory function. Such an analogue may be, for example, Synaptica Peptide with an N-terminal and/or C-terminal extension. It may have at least 80%, at least 90%, at least 95% or more, e.g. 99% homology or identity with the sequence of SEQ. ID. No. I over its entire length or a portion of its length. It may be the in vivo counterpart of Synaptica Peptide.

The term “alpha 7 nicotinic receptor” will be understood to refer to a homomeric receptor of alpha 7 subunits which exhibits in the presence of Ca²⁺ ions and acetylcholine (ACh) induced Ca²⁺ flux which can be (i) blocked by α-bungarotoxin and (ii) modulated by Synaptica Peptide. Such a receptor may be a native homomeric alpha 7 nicotinic receptor in its normal membrane environment. or a homomeric alpha 7 nicotinic receptor inserted into a synthetic membrane or a cellular membrane which does not normally present alpha 7 nicotinic receptors.

A method of the invention as set out above may additionally include the step of determining whether a compound proposed for test, or selected as an agonist or antagonist, is capable of crossing the blood-brain barrier or of formulation for passage across the blood-brain barrier. Antagonists identified by a method as set out above and which are able to pass through the blood-brain barrier are also expected to be capable of antagonising non-enzymatic function of AChE in the brain and hence to be potentially useful therapeutic agents for treatment of neurodegenerative disorders including, for example, as previously indicated above Alzheimer's Disease and Parkinson's Disease.

### BRIEF DESCRIPTION OF THE FIGURES

FIG. 1: Alignment of 5 partial AChE sequences including the Synaptica Peptide sequence, the equivalent regions of 3 butyrylcholinesterases (BChE sequences) and a portion of the human A4 amyloid precursor protein (Hum Amyl). Hum AChE=human AChE; Rab AChE=rabbit AChE; Mus AChE=mouse AChe; Bov AChE=bovine AChE; Hum BChE=human BChE; Rab BChE=rabbit BChE; Mus BChE=mouse BuChE. Residues in bold are conserved across all sequences. Boxed residues are shared by all AChEs and human APP, but by no BChE. The bar above the alignment shows the position of the Synaptica Peptide sequence. The bar below the alignment indicates the homologous region of human APP at the N-terminus of the Aβ1-42 fragment.

FIG. 2: Proposed sequence of events whereby activation of non-enzymatic function of ACheE leads to neurodegeneration in the global neuron population.

FIG. 3: Current-voltage relationship for the human alpha 7 nicotinic receptor presented at the surface of Xenopus oocytes;

FIG. 4: Concentration-response relationship for ACh on human alpha 7 nicotinic receptors presented at the surface of Xenopus oocytes.

FIG. 5: Figure illustrating enhancement by Synaptica Peptide of the ACh-induced Ca²⁺ flux through human alpha 7 nicotinic receptors expressed by Xenopus oocytes when there is no pre-incubation with the peptide.

FIG. 6: illustrating inhibition of the ACh induced Ca²⁺ flux through human alpha 7 nicotinic receptors expressed by Xenopus oocytes when the oocytes are preincubated with the peptide for 2 minutes.

FIG. 7: showing the effect of different concentrations of Synaptica Peptide on ACh currents through human alpha 7 nicotinic receptors expressed by Xenopus oocytes when the oocytes are either not pre-incubated with the peptide or incubated with the peptide for 2 minutes.

FIG. 8: Intracellular recordings in..Xenopus oocytes expressing human alpha 7 nicotinic receptors in the presence of(i)100 μM ACh (ii) 10 μM Synaptica Peptide and (iii) 10 μM Synaptica Peptide when followed by addition of 100 μM ACh after 30 seconds.

FIG. 9 shows dose-dependent decrease in ¹²⁵I-α-bungarotoxin binding to human SH-SY5Y neuroblastoma cells in the presence of biotinylated and amidated Synaptica Peptide.

FIG. 10 illustrates decrease of viability of human SH-SY5Y neuroblastoma cells in the presence of biotinylated and amidated Synaptica Peptide.

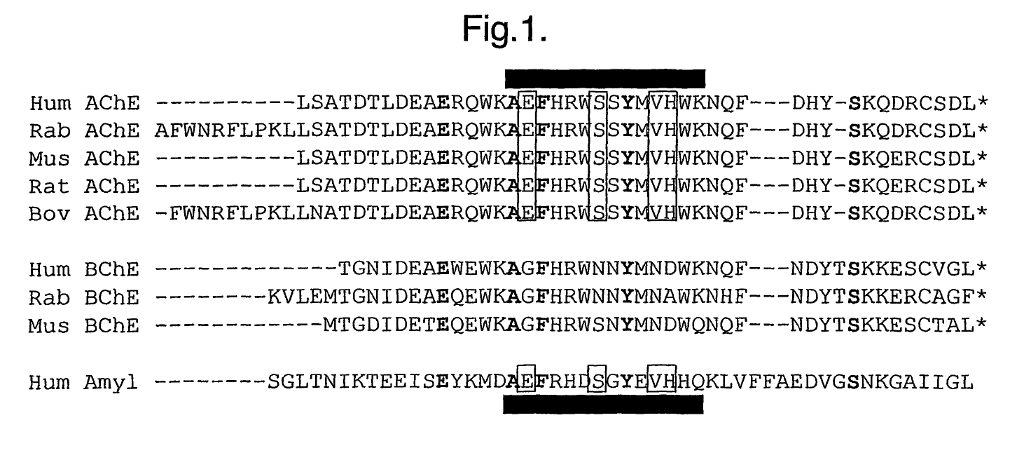

### DETAILED DESCRIPTION OF THE INVENTION

It will be appreciated that screening assays according to the invention may follow a number of protocols. Native alpha 7 nicotinic receptors may be employed without isolation from their normal membrane surrounding, e.g. in the form of a brain tissue slice maintained in vitro, an organotypic tissue culture comprising neuronal cells such as neonate hippocampal cells or cultured cells. Suitable cultured cells for this purpose include, for example, cultured PC-12 cells (rat pheochromocytoma cells) which have previously been shown to express functional α-bungarotoxin-sensitive alpha 7 nicotinic receptors composed of homomers of alpha 7 subunits (Blumenthal et al., J. Neurosci. (1997) 17, 6094-6104, Rangwala et al., J. Neurosci. (1997) 17, 8201-8212 and Drisdel and Green, J. Neurosci. (2000) 20, 133-139). Alternatively, for example, cultured human SH-SY5Y neuroblastoma cells may be employed either expressing their normal level of autologous alpha 7 nicotinic receptor or after transformation to increase alpha 7 nicotinic receptor expression, e.g. by providing additional expression of a heterologous alpha 7 nicotinic receptor as described in Puchacz et al., FEBS Let. (1994) 354, 155-159. Where native alpha 7 nicotinic receptors are employed without isolation from their normal membrane environment, binding of Synaptica Peptide or the test compound to such receptors and/or change of ion flux as a result of such binding will be shown by use of an alpha 7 nicotinic receptor blocker, preferably α-bungarotoxin or a suitable antibody.

Alternatively, however, and preferably alpha 7 nicotinic receptors or functional analogues thereof may be employed inserted into a synthetic membrane or presented at the surface of cells or membrane preparations derived therefrom which do not normally present alpha 7 nicotinic receptors. .Such cells will preferably be derived from cells which do not normally express any Ca²⁺ permeable receptor. They may, for example, be human cells, e.g. human embryonic kidney (BEK) 293 cells, engineered to express alpha 7 nicotinic receptors or functional analogues thereof, e.g. human alpha 7 nicotinic receptors as described in Published International Application WO 94/20617.

Particularly preferred for this purpose are oocytes, e.g. Xenopus oocytes, engineered to express at the cell surface alpha 7 nicotinic receptors or functional analogues thereof. Preparation and maintenance in vitro of such oocytes expressing rat alpha 7 receptor subunits may be, carried out as described in Seguela et al., J. Neurosci., (1993)13, 596-604. Example 1 below describes the production of Xenopus oocytes expressing at the cell surface human alpha 7 subunits as functional α-bungarotoxin-sensitive alpha 7 nicotinic receptors. Preparation of oocytes expressing such functional homomeric receptors is also described in WO 94/20617. Oocytes expressing alpha 7 nicotinic receptors of other species may be prepared in similar manner.

It will be appreciated that by employing, for example, mutant alpha 7 receptor subunit mRNAs for transfection, oocytes may also be prepared in similar manner expressing functional analogues of native alpha 7 receptors. Such functional analogues include receptors formed from chimeric subunits in which the extracellular domain portion of an alpha 7 subunit is joined to a portion of another protein capable of inserting into the cell membrane such that induction of Ca²⁺ influx in the presence of acetylcholine, or another agonist for the acetylcholine binding site of native alpha 7 nicotinic receptors, is maintained, and can be modulated by Synaptica Peptide.

Functional analogues of alpha 7 nicotinic receptors suitable for use in a method of the invention may also be non-homomeric receptors which include 1 or more alpha 7 subunits and retain the following characteristics: (i) induction of Ca²⁺ flux in the presence of ACh, (ii) blockage of such Ca²⁺ flux by α-bungarotoxin at appropriate dose and (iii) modulation of such Ca²⁺ flux by Synaptica Peptide.

Induction of ion permeability through alpha 7 nicotinic receptors for the purpose of screening according to the invention may be achieved in any known manner for opening the calcium channel of such receptors. Preferably, acetylcholine or an alternative agonist capable of binding at the acetylcholine binding site will be employed. It may be particularly preferred to employ choline since alpha 7 nicotinic receptors have been found to have higher affinity for choline than α4β2 receptors and other nicotinic receptors. The anabasine analogue GTS-21 (2,4-dimethoxybenzylidene anabaseine) may alternatively be employed which has been reported to have high functional selectivity for homomeric alpha 7 nicotinic receptors compared to α4β2 nicotinic receptors.

As a preferred embodiment of the invention, there is provided a method of determining the ability of a compound to act as antagonist of the polypeptide of SEQ. ID. No. 1 (Synaptica Peptide) which comprises:

(i) contacting said compound with an alpha 7 nicotinic receptor or a functional analogue thereof in the presence of Synaptica Peptide or a functional analogue of said peptide under condition, whereby in the absence of said compound said peptide or functional analogue thereof modulates induced ion-flux through the receptor and

(iii) determining whether said compound antagonises the modulatory effect of said peptide or functional analogue thereof on the induced ion-flux,

wherein if said receptor is a native alpha 7 nicotinic receptor in its normal membrane environment, it is identified by means of inhibition by an alpha 7 nicotinic receptor blocker

The ion flux which is determined in such a method may be Ca²⁺ ion flux. However, it will be appreciated that other ions capable of passing through the calcium channel of alpha 7 nicotinic receptors may be employed, e.g. ⁸⁶Rb ion flux may be determined as described in Published International Applications WO 91/15602 and WO 94/20617. The modulatory effect observed in the absence of the test compound may be enhancement and/or reduction of the ion flux depending on the dosage and time of application of Synaptica Peptide or the functional analogue thereof.

Thus, a particularly preferred embodiment of the invention is a method for determining an antagonist of Synaptica Peptide which comprises:

(i) providing cultured cells, preferably oocytes, engineered to express alpha 7 nicotinic receptors or functional analogues thereof at their outer surface in a medium containing Ca² said cells not expressing any other Ca²⁺ permeable receptor;

(ii) contacting said cells with (a) the compound to be tested, (b) means to induce Ca²⁺ flux through said receptor, or analogues, preferably acetylcholine or an alternative agonist capable of binding at the acetylcholine binding site of said receptors or analogues in an amount sufficient to induce Ca²⁺ permeability and (c) Synaptica Peptide or a functional analogue thereof in an amount sufficient to modulate Ca²⁺ through said receptors; and

(iii) determining. whether the test compound inhibits enhancement or reduction of Ca²⁺ flux observed in the presence of said peptide or analogue minus the test compound.

Synaptica Peptide or the functional analogue thereof will preferably be added to the culture medium so that there is no pre-incubation with the peptide or analogue prior to induction of Ca²⁺ flux and at a dose which initially produces enhancement of Ca₂₊ flux in the absence of the test compound. A suitable concentration of Synaptica Peptide or the chosen functional analogue thereof may be readily determined by initial experimentation. A concentration of Synaptica Peptide of about 0.001 μM has been found suitable with Xenopus oocytes expressing human alpha 7 nicotinic receptors (see Example 1). Suitable concentrations of agonist for Ca²⁺ channel opening may also be readily determined. A concentration of acetylcholine of about 50 μM to 100 μM has been found suitable with Xenopus oocytes expressing human alpha 7 nicotinic receptors (see Example 1). Calcium influx can be followed by measuring change in membrane potential or by detection of intracellular Ca²⁺ ions using a Ca²⁺ detection agent such as fura and UV/visible or fluorescence spectroscopy. As indicated above, instead of Ca²⁺ ions, any other nicotinic receptor -permeable ions may alternatively be employed including Na⁺, K⁺, Ba²⁺ and ⁸⁶Rb ions.

Such a test protocol can, of course, be modified to alternatively identify a functional analogue of Synaptica Peptide, e.g. a functional variant of Synaptica Peptide. In this case, cells expressing alpha 7 nicotinic receptors or functional analogues thereof will be incubated with the compound under test in the absence of Synaptica Peptide or a functional analogue thereof and means provided to induce ion permeability. The compound will be capable of competitively binding with Synaptica Peptide to the chosen target receptors. Enhancement or reduction of ion influx compared to that observed in the absence of the test compound, which can be blocked by an alpha 7 receptor blocker such as α-bungarotoxin or a specific antibody, is indicative of a compound capable of mimicking the action of Synaptica Peptide at its modulatory site on the alpha 7 nicotinic receptor. Again, the cells employed will preferably be cells which express at their outer surface alpha 7 nicotinic receptors or functional analogues thereof in the absence of other Ca²⁺ permeable receptors, most preferably oocytes expressing alpha 7 nicotinic receptors or functional analogues thereof.

Identification of functional analogues and antagonists of Synaptica Peptide in accordance with the invention may also take the form of a neurite growth assay employing cultured neuronal cells upon which Synaptica Peptide can produce a toxic effect dependent on dosage and exposure time mediated via action on alpha 7 nicotinic receptors. Such cells are exemplified by GABA positive neurites in an organotypic tissue culture of neonate hippocampus, e.g. rat neonate hippocampus (see Example 2). Thus in a further embodiment, the present invention provides a method of identifying an antagonist of biological activity of Synaptica Peptide which comprises:

(i) providing cultured neurites upon which Synaptica Peptide is capable of producing a toxic effect dependent upon dose and exposure time;

(ii) incubating said neurites (a) “in the presence of Synaptica Peptide or a functional analogue thereof at a dose sufficient to produce said toxic effect, (b) in the presence of the same dose of Synaptica Peptide or the chosen functional analogue thereof and the compound to be tested and (c) in the presence of the same dose of Synaptica Peptide or the chosen functional analogue thereof and an alpha 7 nicotinic receptor blocker such as. α-bungarotoxin at a dose sufficient to block alpha 7 nicotinic receptors in said neurites; and

(iii) observing after a predetermined time whether said test compound inhibits a toxic effect of Synaptica Peptide on said neurites which is prevented by said blocker.

In step (iii), neurite ougrowth can be measured by an increase in the density of neurites and/or an increase in neurite length. An antibody may be employed to identify the neurites of interest. As indicated above, the cultured neurites may, for example, be GABA positive neurites present in an organotypic tissue culture of neonate hippocampus. In this case, it will be found convenient, for example, to treat the tissue culture with the biotinylated and amidated analogue of Synaptica Peptide (see Table 1 in Example 2).

A modification of such a neurite growth assay may be employed to identify functional analogues of Synaptica Peptide. Thus, the present invention also provides a method of identifying a functional analogue of Synaptica Peptide, e.g. a functional variant thereof,. which comprises:

(i) providing cultured neurites upon which Synaptica Peptide is capable of producing a toxic effect dependent upon dose and exposure time;

(ii) treating said neurites with (a) the compound to be tested and (b) the compound to be tested and an alpha 7 nicotinic receptor blocker at a dose sufficient to block alpha 7 nicotinic receptors in said neurites; and

(iii) determining whether the test compound produces a toxic effect on said neurites which is prevented by said blocker, said compound being capable of competitively binding with Synaptica Peptide to said receptors.

In a further aspect of the invention, there is provided a method for determining the ability of a compound to act as an antagonist of Synaptica Peptide, which comprises determining whether said compound can inhibit the action of Synaptica Peptide or a functional analogue thereof on alpha 7 nicotinic receptors bound to a support or presented at the surface of cells, e.g. human SH-SY5Y neuroblastoma cells, or cell membranes. Such a method may, for example, take the form of a binding assay and/or a cell viability assay (see Example 3). Compounds may, for example, be screened for (i) ability to inhibit decrease in binding of α-bungarotoxin or a functionally equivalent protein to the cells in the presence of Synaptica Peptide or a functional analogue thereof and/or (ii) ability. to inhibit decrease of cell viability in the presence of Synaptica Peptide or a functional analogue thereof. Compounds may alternatively be screened for ability to mimic such effects of Synaptica Peptide on alpha 7 nicotinic receptors. Again, it will be appreciated that such compounds which represent functional analogues of

Synaptica Peptide will be capable of competitively binding with Synaptica Peptide to alpha 7 nicotinic receptors, for example such receptors presented by cells.

When screening for functional analogues or antagonists of Synaptica Peptide, it may be deemed desirable, or necessary, to initially screen for compounds capable of competitively binding with Synaptica Peptide to alpha 7 nicotinic receptors by a competitive binding assay. It will suffice for this purpose to employ alpha.7 nicotinic receptors or derivatives thereof, including individual alpha 7 receptor subunits or modifications thereof and portions of such subunits, provided binding ability. for Synaptica Peptide is retained. Alpha 7 nicotinic receptors or derivatives thereof may be employed in membrane-bound form, e.g. as part of whole cells or an isolated membrane preparation, or in non-membrane bound form. Alpha 7 nicotinic receptors may be solubilised from membranes and isolated by affinity purification using α-bungarotoxin-conjugated Sepharose 4B as described by Drisdel and Green (ibid).

In a still further aspect, the present invention provides a method for screening a compound for ability to interact with an alpha 7 nicotinic receptor, which comprises contacting said compound with said receptor or a derivative thereof which retains a modulatory binding site for Synaptica Peptide in the presence of Synaptica Peptide or a functional peptide variant thereof and determining whether binding of Synaptica Peptide or the variant thereof is inhibited or prevented.

Preferably, the alpha 7 nicotinic receptor or derivative thereof will be non-membrane bound or presented by whole cells or a membrane preparation modified to present the target protein. For use in such a competitive binding assay, Synaptica Peptide or a variant thereof may be labelled with a revealing label for direct detection. Such a label may be any label conventionally employed for labelling proteins for detection, including a radioactive label, a fluorescence label, an enzyme label or detectable non-enzyme label such as biotin or an ESR or NMR detectable label. Such an assay may take the form of an alpha 7 nicotinic receptor pull-down assay in which Synaptica Peptide or a derivative thereof is labelled with a ligand such as biotin and beads such as magnetic beads are provided linked to a receptor for said ligand. Such an assay may comprise the steps of:

(i) providing a preparation of alpha 7 nicotinic receptors, or derivatives thereof which retain a modulatory binding site for Synaptica Peptide, in non-membrane bound form or in the form of a cell lysate, e.g a neonate rat hippocampal cell lysate;

(ii) contacting said preparation with (a) Synaptica Peptide or a derivative thereof labelled with a ligand such that it retains ability to bind to the said modulatory site and (b) the compound to be tested;

(iii) additionally providing beads, e.g magnetic beads, linked to a receptor for said ligand label;

(iv) separating the beads from the preparation and determining whether there is reduction in the amount of captured alpha 7 nicotinic receptor or derivative thereof compared to the same assay carried out in the absence of the test compound.

Determination of any captured receptor or derivative thereof may take the form of Western blotting employing an antibody capable of identifying the receptor or derivative. Anti-alpha 7 antibodies for this purpose may be prepared by conventional techniques for obtaining antibodies to specific proteins and are obtainable from a number of sources. For example, a goat polyclonal anti-alpha 7 antibody is commercially available from Santa Cruz Biotechnology

In a still further aspect, the present invention provides a method of preparing a functional analogue or antagonist of Synaptica Peptide which comprises:

(i) identifying said functional analogue or antagonist by a method of the invention as described above and

(ii) synthesising said functional analogue or antagonist.

In the case of a compound so prepared, synthesis of the compound may be followed by incorporation into a pharmaceutical composition. The compound may be formulated together with a pharmaceutically acceptable carrier or diluent for passage across the blood-brain barrier. Means for such formulation may be conventional means well known in the pharmaceutical art.

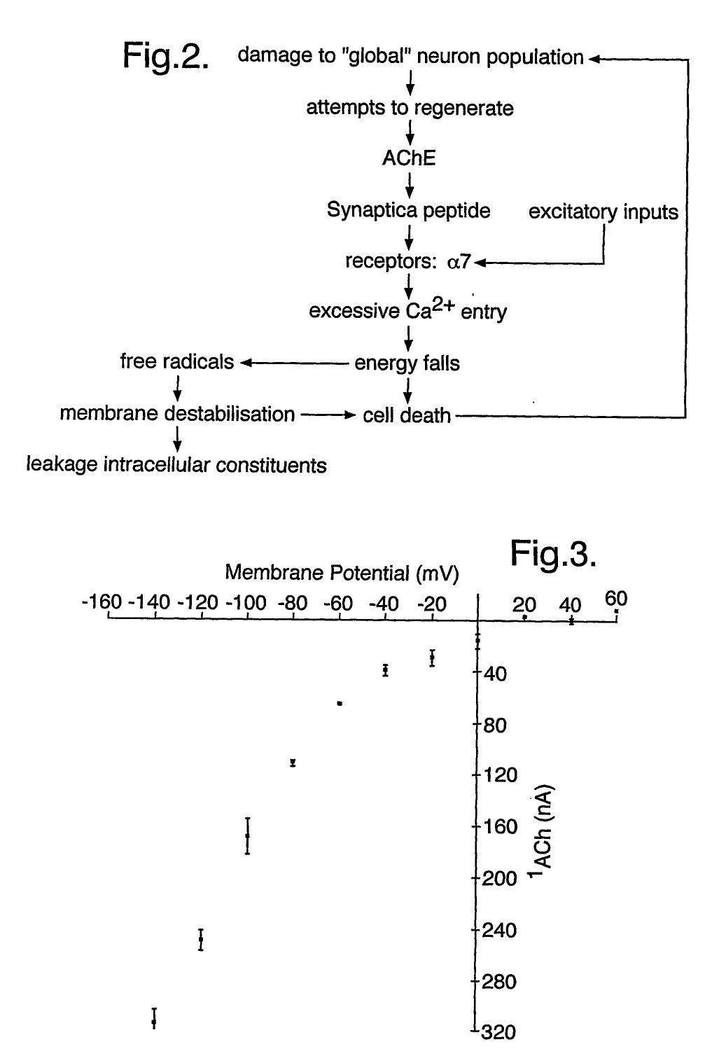

The present invention also provides functional analogues and antagonists of Synaptica Peptide identified by a screening protocol of the invention and pharmaceutical compositions containing such analogues and antagonists together with a pharmaceutically acceptable carrier or diluent. As previously indicated, particularly preferred, for example, are antagonists thus identified which are capable of formulation for passage through the blood-brain barrier and thus inhibiting or preventing toxic non-enzymatic activity of AChE in vivo. Such compounds are envisaged as highly advantageous therapeutic agents for neurodegenerative disorders in view of their high selectivity for areas of the brain which may be affected by non-enzymatic function of AChE and ability to arrest or inhibit cell loss (see FIG. 2 which summarises the proposed sequence of events leading to neuronal degeneration arising from activation of non-enzymatic activity of AChE).

In a still further aspect of the invention, there is thus provided use of an antagonist of Synaptica Peptide identified as described above for use in the preparation of a medicament for use in the treatment of a neurological disorder associated with non-enzymatic function of AChE, especially, for example, Alzheimer's Disease, Parkinson's disease or Motor Neuron Disease.

The following examples illustrate the invention.

### EXAMPLES

### Example 1

Modulation by Synaptica Peptide of Ca²⁺ Flux Through Alpha 7 Nicotinic Receptors at the Surface of Oocytes

Summary

With Xenopus oocytes transfected with alpha 7 subunit mRNA, a dose dependent modulatory effect can be observed on Ca²⁺ flux induced by acetylcholine. Synaptica Peptide alone has no effect on Ca²⁺ permeability. In the absence of pre-incubation with Synaptica Peptide, Ca²⁺ flux induced by acetylcholine may be observed to be initially enhanced followed by shutting off of Ca²⁺ channels. Such observations are indicative of Synaptica Peptide binding at a modulatory site on the alpha 7 nicotinic receptor since there is no other site on the external surface of the oocytes with which the peptide might be interacting.

Experimental Procedures

Preparation of RNA Transcripts and Xenopus Oocyte Injection

Complementary DNA encoding for the human alpha 7 nicotinic acetylcholine receptor was obtained as described by Peng et al. (Human alpha 7 acetylcholine receptor: cloning of the alpha7 subunit from the SH-SY5Y cell line and determination of pharmacological properties of native receptors and functional alpha 7 homomers expressed in Xenopus oocytes (1994) Molecular Pharmacology, 45, 546-554). Total RNA was isolated from the human neuroblastoma cell line SH-SY5Y (purchased from the European Tissue Culture bank, UK) and apoly-(A)+RNA was isolated and used to construct a lambdaZap II cDNA library. Several million plaques were screened with a human alpha 7 cDNA probe. Hybridizations were performed at 42° C. in 40% formamide, 5x Denhardt's solution, 0.5% sodium dodecyl sulphate, 5x (0.18 M NaCl, 0.01 M sodium phosphate buffer, pH 7.4, 1 mM EDTA), 0.15 mg/ml denatured salmon sperm DNA. Membranes were washed and then exposed to Kodak XAR film. The selected clone was sequenced and its sequence was shown to be identical to that. registered by Peng et al. (EMBL accession number X70297).

Human alpha 7 receptor cDNA was ligated into a plasmid vector under the control of an SP6 promoter and in vitro transcribed alpha 7 subunit MRNA was used to transfect Xenopus oocytes. The RNA synthesis mixture was diluted (1:50) into RNA-free distilled water and then injected into stage V-VI Xenopus oocytes, which had been isolated and defolliculated manually. Injected oocytes were maintained for up to a week at 18° C. in Barth's solution at pH 7.2 containing (mM): 88 NaCl, 1 KCl, 0.41 CaCl₂, 0.82 MgSO₄, 0.33 Ca(NO₃), 2.5 NaHCO₃,0.5 theophylle, 10 HEPES plus 0.1 μg/ml⁻¹ gentamicin sulphate, 0.01 mg/ml⁻¹ streptomycin sulphate and 0.01 mg/ml⁻¹ penicillin-G.

Electrophysiology

Recordings were made 1 to 7 days following injection of RNA and at room temperature. 15 Whole cell currents were measured by two microlectrode voltage clamp (Geneclamp 500B, Axon Instruments) using 0.6-2.5 MΩ agarose-cushioned electrodes containing 3 M KCl. Oocytes were placed in a 100 μl bath, which was gravity perfused continuously at 4 ml/min with Rinaer's solution (mM: 115 NaCl, 2.5 KCl, 10 HEPES, 1.8 BaCl, pH 7.2).Agents to be applied to the oocytes were dissolved in the perfusion solution and applied by gravity perfusion using a manually-activated valve.

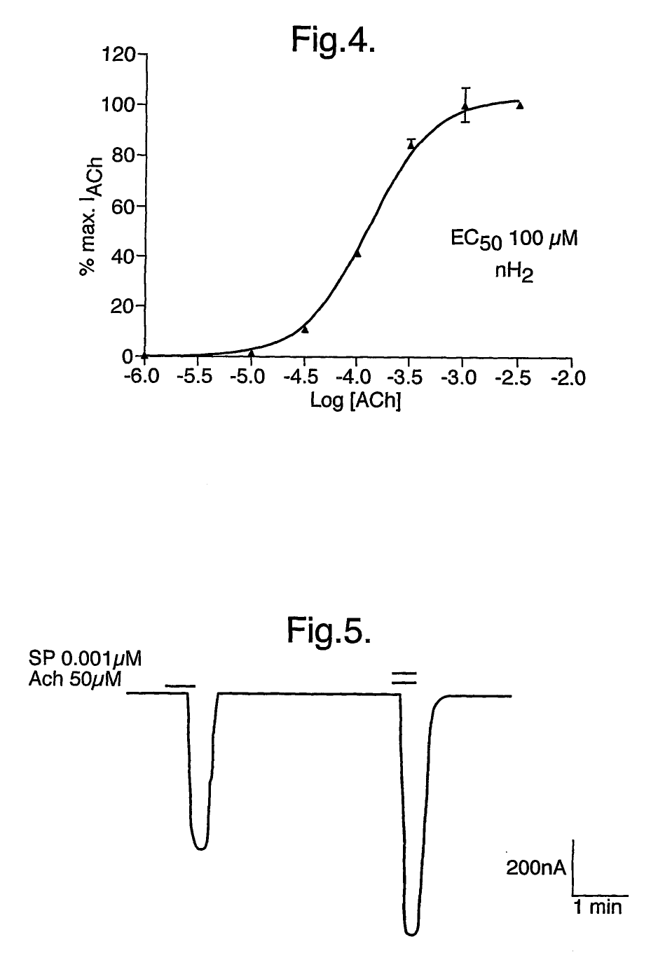

Functional alpha 7 nicotinic receptors were shown to be expressed after 24 hours post-injection of mRNA by monitoring ACh-induced currents. ACh currents were comparable with those mediated by native alpha 7 nicotinic receptors in their normal membrane environment (currents rectify inwardly, EC₅₀ for ACh is around 100 μM with a Hill coefficient (nH) of 2; see FIGS. 3 and 4). 50 μM or 100 μM ACh was used for all tests with Synaptica Peptide.

Treatments with Synaptica Peptide

Application of Synaptica Peptide on its own to oocytes presenting alpha 7 nicotinic receptors did not modify whole membrane currents. When Synaptica Peptide was applied to such oocytes at 0.001 μM with ACh, increased Ca²⁺ flux was indicated (see FIG. 5).

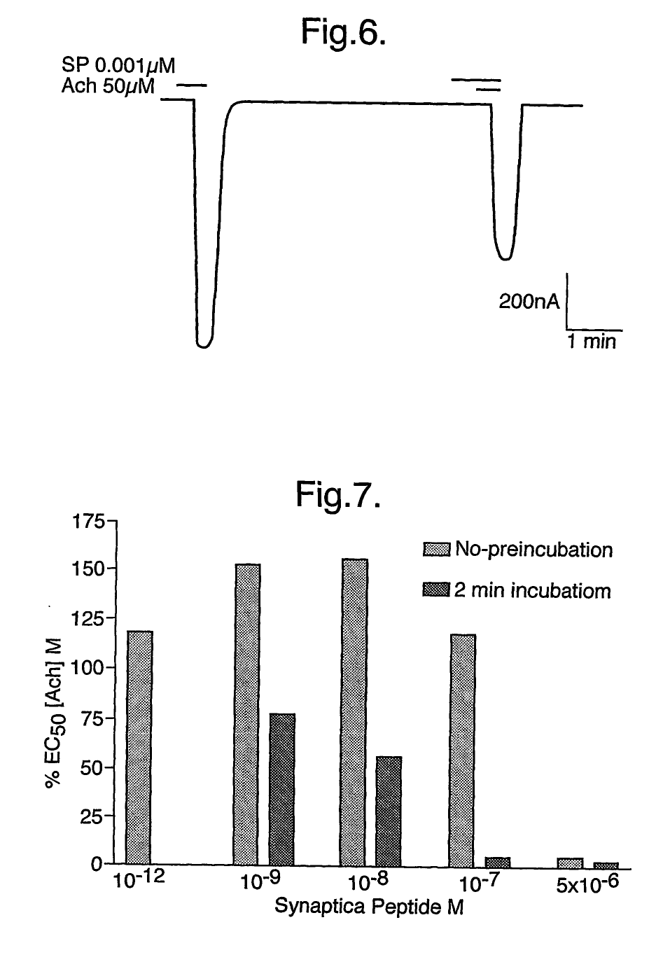

Application of Synaptica Peptide followed after 2 minutes by co-application of Synaptica Peptide and ACh caused inhibition of ACh-mediated responses (see FIG. 6). Further experiments were carried out with application of Synaptica Peptide at different concentrations in the range 10⁻¹² to 5×10⁻⁶ with no pre-incubation or pre-incubation for 2 minutes (see FIG. 7). When a high dose of Synaptica Peptide was employed, the enhancement of ACh-evoked response observed with low doses of the peptide was reversed. This trend occurred at lower concentrations of Synaptica Peptide if the peptide was given 2 minutes before application of ACh thereby effectively increasing the local concentration. Synaptica Peptide effects were found to be reversible but recovery was slow. These results are consistent with Synaptica Peptide acting at a modulatory site on alpha 7 nicotinic receptors and are also consistent with previous studies of the effect of Synaptica Peptide on ACh-induced calcium entry into neurons of neonate. rat hippocampus and substantia nigra cultured in vitro.

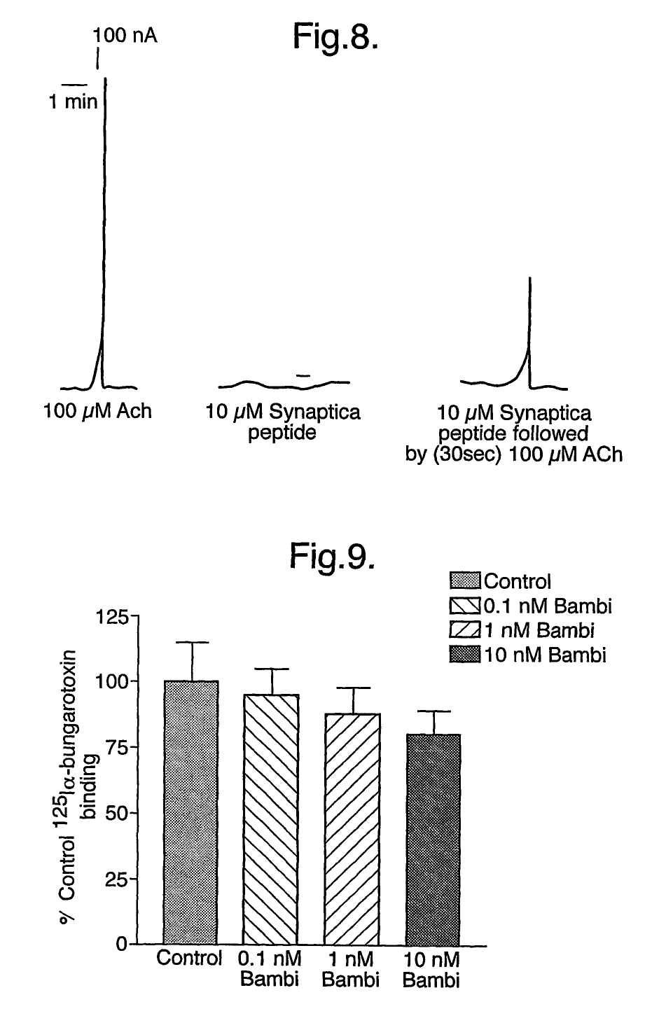

FIG. 8 shows additional data obtained by intracellular recordings in Xenopus oocytes expressing human alpha 7 nicotinic receptors. In these studies, intracellular recordings with application of 100 μM ACh showed as expected opening of Ca²⁺ channels. When 10 μM of Synaptica Peptide was applied without ACh, no Ca²⁺ current was observed. Addition of 10 μM Synaptica Peptide followed by after 30 seconds 100 μM ACh gave a reduced current compared to that observed with ACh alone. This is consistent with so much calcium. entering the neurons, the calcium channels are shut off leading to reduction in the observed current.

Identification of Agonists and Antagonists for the Modulatory Action of Synaptica Peptide on Alpha 7 Nicotinic Receptors

The protocols described above with Xenopus oocytes may be modified to provide an advantageous system for identify functional analogues and antagonists of Synaptica Peptide. For example, a test compound may be substituted for Synaptica Peptide to determine if it will mimic the action of Synaptica Peptide on the alpha 7 nicotinic receptors. Alternatively, the test compound may. be added with Synaptica Peptide to determine whether it will inhibit the effect of Synaptica Peptide in enhancing or inhibiting ACh-induced Ca²⁺ flux.

### Example 2

Action of BamBi Peptide on GABA-Containing Neurons of Neonatal Hippocampus

Summary

In organotypic tissue culture of neonatal rat hippocampus, the biotinylated and amidated version of Synaptica Peptide (BamBi Peptide) has been found to have a significant toxic action on GABA-containing neurons.

Experimental Procedures

(i) Isolation of hippocampal Slices

Hippocampal slices were prepared from 7 day old Wistar rats. This age was chosen as the peak of neuronal migration has passed and the cytoarchitecture of the brain is already established, the brain is large enough for ready dissection and also brain nerve cells can survive explantation. Rats were decapitated by a scissor cut at the level of the foramen magnum. The skull was then cut along the midline from the base to the front and two horizontal cuts were made at the level of the ears. The skull was peeled away, cranial nerves cut and the brain removed and placed on the dorsal surface on a sterile petri dish on ice. A longitudinal cut was made through the medial cortex following the borders of the hippocampus caudally and the cortex was folded aside. A single cut was made through the cingulate cortex and underlying fimbria to prise the hippocampus free of the thalmus. Excess cortex and ahering meninges were removed before the hippocampus was placed ventral surface down on a McIlwain tissue chopper. Coronal sections (400 μm) were cut and individual slices were separated with the aid of a dissecting microscope and fine spatulas. These slices were placed at 4° C. in fresh filtered Dulbecco's minimal essential medium SAEMD ) for 1.5 hours. Leaving the slices in a balanced salt solution at 4° C. allowed tissue debris and potentially toxic substances such as excitory amino acids to diffuse away and ruptured cell membranes to reseal.

(ii) Mounting of the Tissue Sections

400 μM hippocampal slices were attached to cleaned sterile poly-d-lysine coated coverslips by means of a plasma clot, formed by mixing a solution of chicken plasma (lyophilised chicken plasma 20 μl, reconstituted in 5 ml distilled water) with bovine thrombin (0.8 mg/ml). The tissue section was placed on a 25 μl drop of plasma on a coverslip and a 20 μl drop of thrombin was then placed adjacent to the plasma drop. The two solutions were gently mixed in a circular manner until the clot covered the entire coverslip with the section held in the centre. After mounting the sections, the coverslips were refrigerated for 1.5 hours to allow the clot to set.

(iii) Maintaining Organotypic Slice Cultures

Once the plasma clots had set thus holding the hippocampal slices in place, each coverslip was placed in a diagonal sided tube with a vented lid to assure adequate aeration avoid necrosis. Serum containing media (200 μl) was added to each tube and the tubes placed horizontally in a humidified incubator (37° C., 5% CO₂/95% O₂) for 48 hours.

Following this period, the medium was replaced with 500 μl of fresh filtered serum containing media and cultures were placed on a roller drum and replaced in the incubator at a 5° angle rotating at 10 revolutions per hour. This positioning and rotation of the cultures was important for two reasons. First, the cultures spent half the incubation time exposed to culture media and half to air, thus ensuring adequate aeration, Secondly, the rotation was important for thinning the cultures.

Cytostatic solutions were added to the media for the entire cultivation period to prevent. over-proliferation of non-neuronal cells. The anti-mitotic substances use were uridine and cytosine-β-D-furanoside, both at 1 mM. Culture media was changed once a week by tipping out the old media and replacing it with 500 μl of fresh filtered media warmed to 37° C. Cultivation was continued for 14 days prior to treatment.

(iv) Synthesis of BamBi Peptide

BamBi peptide (1 μM) was prepared by solid phase synthesis employing amide resin from Novabiochem. Biotin was added using HBTU as a coupling reagent and “easylink” NHSbiotin from Pierce & Warriner.

(v) Treatments BamBi Peptide (1 μM) either in the absence of α-bungarotoxin or in the presence of α-bungarotoxin (1 μLM) was added in serum-free media following a period of 24 hours serum starvation. Treatments were for 1 hour to 14 days at 37° C. on the roller drum.

(vi) Immunocytochemical Identification of a Microtubule-Associated Protein (MAP-2) and GABA Positive Neurons

The microtubule-associated proteins (MAPs) of brain exhibit various properties that suggest that they are important in the growth and stabilization of axons and dendrites during neuronal morphogenesis. MAP-2 is highly concentrated in neurons as one of the major components of microtubules and so is used as a stringent marker for neurons in the central nervous system.

By immunocytochemistry, it has been shown that in adult rat two high molecular weight forms of MAP-2 (MAP-2a and MAP-2b) are abundant in neuronal perikaraya and dendrite. A third splice variant (MAP-2c) is absent in the adult but represents ajuvenile form that is abundant during embryonic life. MAP-2a, MAP-2b and MAP-2c are all present at birth, although during subsequent weeks MAP-2b increases transiently reflecting increased dendritic growth, After this critical stage of development, MAP-2c finally disappears towards adulthood.

A monoclonal anti-MAP-2 (Roche) which specifically identifies MAP-2a and MAP-2b was used to measure dendrite length in treated tissue cultures. An anti-glutamic acid decarboxylase antibody (GAD monoclonal antibody; Chemicon) was use to identify GABA positive neurons.

On completion of the incubation period (14 day cultivation period and treatment period), immunocytochemical staining for MAP-2 and/or GAD was performed according to the biotin-avidin-peroxidase method. Cultures were cultivated for 14 days. before being fixed in 4% formaldehyde for I hour at room temperature. In order. to block any non-specific binding sites, cultures were incubated in 20% goat serum in PBS for 1 hour at room temperature. Monoclonal antibodies were added to the cultures (100 μl/culture in PBS plus 0.1% TritonX-100) lying in ahumid chamber for 24 hours at 4° C. and then incubated with biotinylated secondary (100μl/culture) for 1 hour at room temperature. Hydrogen peroxide was used to eliminate any endogenous peroxidase activity (0.3% hydrogen peroxide, 10% methanol in PBS) before finally cultures were incubated in avidin-biotin complex (ABC elite kit) and antibody detected using a diaminobenzidine (DAB) chromogen (0.05% DAB, 0.015% hydrogen peroxide in PBS).

Light microscopy was used to calculate neurite outgrowth measurements and maximal neurite outgrowth was measured for 10 neurites from each culture. Recordings were averaged for each culture and within each culture group and expressed as a percentage of the media only group. Results from treatment groups were taken from at least 3 different culture days (i.e. made from different litters on different days). N numbers for stastical analysis were 11 for each group. Statistical analysis was by means of a one way Anova and a post-hoc Dunnetts mutiple comparison test.

Results

Table 1 below shows the effect of Bambi Peptide on GABA neurons in rat neonate hippocampus at various concentrations and treatment times.

TABLE 1

| BamBi Peptide | Time of treatment | | | | | |
|---|---|---|---|---|---|---|
| conc. (M) | 1 hr | 24 hrs | 48 hrs | 72 hrs | 7 days | 14 days |
| 10⁻¹² | ns | — | — | — | — | — |
| 10⁻⁹ | pre-a* | pre-a | ns | ns | apo# | apo |
| 10⁻⁸ | pre-a | pre-a | ns | ns | apo | apo |
| 10⁻⁷ | — | apo | apo | apo | apo | apo |
| 10⁻⁶ | pre-a | apo | apo | apo | apo | apo |
| 10⁻⁵ | necrotic | necrotic | necrotic | necrotic | necrotic | necrotic |

*pre-a = pre-apoptotic
#apo = apototic

Use of Hippocampal GABA Neurons for Identifying Agonists and Antagonists of Synaptica Peptide

The protocol discussed above can be adapted to identify functional analogues and antagonists of Synaptica Peptide. For this purpose, organotypic tissue cultures of rat neonatal hippocampus will be prepared in the same manner: as above. For determination of a functional analogue of Synaptica Peptide, cultures will be treated with the test compound or the test compound together with α-bungarotoxin at a dose sufficient to block alpha 7 nicotinic receptors. Observation of toxic action on GABA neurons in the presence of the test compound, which is prevented by α-bungarotoxin, will be indicative of a functional analogue acting at the modulatory binding site for Synaptica Peptide on the alpha 7 nicotinic receptor.

For determination of a Synaptica Peptide antagonist, cultures will be treated with (a) BamBi Peptide alone at a dose sufficient to give a toxic action on GABA neurons (b) the same dose of BamBi peptide and α-bungarotoxin at a dose sufficient to block alpha 7 nicotinic receptors and (c) the same dose of BamBi Peptide supplemented with the test compound.

Synaptica peptide may be similarly” employed in neurite outgrowth assays.

### Example 3

Binding of Synaptica Peptide (Biotinylated and Amidated) to Alpha 7 Nicotinic Receptors on human SH-SY5Y Neuroblastoma Cells

SH-SY5Y European Tissue Culture, IU K) clonal cells were grown to confluence and then treated with biotinylated and amidated Synaptica Peptide (BamBi) at a range of concentrations (10⁻¹²-10⁻⁸ M) for three days, after which tine they were harvested with sterile Phosphate Buffered Saline (PBS) and centrifuged at 240 g for 2 minutes. Pellets were then resuspended in 1 ml binding buffer (BB) consisting of 140 mM. NaCl, 1 mM EDTA and 50 Tris-HCl at pH 7.4. Cells were counted using a haemocytometer and diluted in BB to give a final concentration of 2,000 000 cells per assay tube.

Binding Assay Protocol

All experiments were carried out with 100 μl of whole cell suspension and 50 μl of ¹²⁵I-α-bungarotoxin in a total volume of 250 μl. Non-specific binding was determined using 1 mM nicotine. For equilibrium competition binding assays, 2 nM ¹²⁵I-α-bungarotoxin was incubated with BamBi peptide at concentrations ranging from 1 pM to 3 mM. The same BB and cell number were used in saturation binding studies, but the concentration of ¹²⁵I-α-bungarotoxin varied between 0.1 to 5 nM. Cells were pre-incubated with nicotine for 30 minutes prior to the addition of ¹²⁵I-α-bungarotoxin. All reactions were performed in triplicate in borosilicate glass binding tubes and were incubated at room temperature for 90 minutes. Cells were washed twice with 4 ml ice cold binding saline to stop the reaction.. Bound and free fractions were separated by vacuum filtration (Millipore).through glass microfibre filters (CF/C, Whatman). Radioactivity was counted using a gamma counter.

Cell Viability

To determine the effect of BamBi peptide on cell viability, 500,000 cells were seeded onto 12-well culture plates. When the cells reached confluency, 10⁻¹² M to 10⁻⁸ M Bambi peptide was added to the cultures. The cultures were grown in the presence of the peptide for up to 3 days. Cell death induced by BamBi peptide was assessed by tryptan blue dye exclusion. The cell cultures were stained immediately with 1.5% tryptan blue for 10 min at room temperature and then rinsed with physiological saline. Cells stained with tryptan blue were considered nonviable. At least 200 cells were counted to determine viability for each culture well. In each experiment, cell counts on five wells were averaged to obtain the mean±sem.

Results

BamBi peptide was found to produce a dose-dependent decrease in ¹²⁵I-α-bungarotoxin binding to SH-SY5Y cells as shown in FIG. 9. The effect was reversed in the presence of 1 mM nicotine.

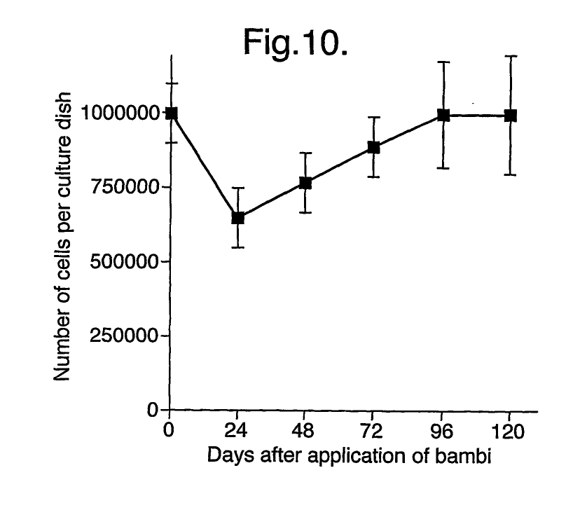

BamBi Peptide produced an acute and significant decrease in cell viability after 24 hours at all concentrations tested. However, this effect was reversible and after 72-90 hours cell numbers were similar to those of control cultures (see FIG. 10).

Use of SH-SY5Y Cells for Identifying Agonists and Antagonists of Synaptica Peptide.

Compounds may be screened for ability to mimic the above-noted effects of Bambi Peptide on SH-SY5Y cells or the ability to inhibit the same effects of Synaptica Peptide or a known functional analogue thereof.

### Example 4

Pull-Down of Alpha 7 Nicotinic Receptors Using Biotin-Labelled Synaptica Peptide

Summary

Biotin-labelled Synaptica Peptide combined with use of magnetic beads carrying steptavidin can be used to pull-down receptors from a rat neonate hippocampal lysate which are recognised by an anti-alpha 7 antibody. This provides further evidence that the target site for the non-enzymic function of AChE as mediated by Synaptica Peptide is the alpha 7 nicotinic receptor.

Experimental Procedure Biotinylated Synaptica Peptide was incubated with streptavidin-coated magnetic beads. The beads were washed to remove unbound peptide and incubated with a rat neonate hippocampal cell lysate. The beads were then washed to remove unbound material and protein eluted therefrom with low pH or SDS. The eluate was run on SDS-PAGE gels and Western blotting carried out using an anti-alpha 7 antibody. Pull down of alpha 7 nicotinic receptors was indicated. Such pull-down was not observed when adult rat hippocampus tissue was used.

1. Use of an alpha 7 nicotinic receptor or a functional analogue thereof to determine whether a compound is capable of acting as a functional analogue or antagonist of the polypeptide of SEQ. ID. No. 1 (Synaptica Peptide) on said receptor, wherein if said receptor is a native alpha 7 nicotinic receptor in its normal membrane environment, it is identified by means of inhibition by: an alpha 7 nicotinic receptor blocker.

2. A method for determining the ability of a compound to act as an * antagonist of the polypeptide of SEQ. D. .No. 1 (Synaptica Peptide), which comprises determining whether said compound can inhibit binding of Synaptica Peptide or a functional analogue thereof to its target site on an alpha 7 nicotinic receptor, or a functional analogue of said receptor, and. thereby antagonise the modulatory effect of Synaptica Peptide or its analogue on induced ion flux through the receptor.

3. A method as claimed in claim 2 for determining the ability of a compound to act as an antagonist of Synaptica Peptide, which comprises:
(i) contacting the test compound with an alpha 7 nicotinic receptor or functional analogue thereof in the presence of Synaptica Peptide or a functional analogue of said peptide under conditions whereby in the absence of the test compound said peptide or functional analogue thereof modulates induced ion-flux through the receptor and
(ii) determining whether the test compound antagonises the modulatory effect of said peptide or functional analogue thereof on the induced ion flux,
wherein if said receptor is a native alpha 7 nicotinic receptor in its normal membrane environment, it is identified by means of inhibition by an alpha 7 nicotinic receptor blocker.

4. A method according to claim 3 wherein ion flux through the alpha 7 nicotinic receptor or functional analogue thereof is induced by acetylcholine or another agonist for opening of the ion channel of said receptor.

5. A method as claimed in claim 4 wherein ion flux through the alpha 7 nicotinic receptor or functional analogue thereof is induced by choline.

6. A use or method as claimed in any one of claims 1 to 4 wherein cultured cells expressing an alpha 7 nicotinic receptor or functional analogue thereof are employed.

7. Amuse or method according to claim 6 wherein said cells do not express any other Ca²⁺ permeable receptor.

8. A use or method according to claim 7 wherein. said cells are oocytes.

9. A method for determining the ability of a compound to act as an antagonist of the polypeptide of SEQ. ID No. 1 (Synaptica Peptide), which comprises:
(i) providing cultured neurites upon which Synaptica Peptide is capable of producing a toxic effect dependent upon dose and exposure time;
(ii) incubating said neurites (a) in the presence of Synaptica Peptide or a functional analogue thereof at a dose sufficient to produce said toxic effect, (b) in the presence of the same dose of Synaptica Peptide or the chosen functional analogue thereof and the compound to be tested and (c) in the presence of the same dose of Synaptica Peptide or the chosen functional analogue thereof and an alpha 7 nicotinic receptor blocker at a dose sufficient to block alpha 7 nicotinic receptors; and
(iii) observing after a predetermined time whether said test compound inhibits a toxic effect of Synaptica Peptide on said neurites which is prevented by said blocker.

10. A method for determining whether a compound is a functional analogue of the polypeptide of SEQ. ID no. 1 (Synaptica Peptide), which comprises:
(i) providing cultured neurites upon which Synaptica Peptide is capable of producing a toxic effect dependent upon dose and exposure time;
(ii) incubating said neurites with (a) the compound to be tested and (b) the compound to be tested and an alpha 7 nicotinic receptor blocker at a dose sufficient to block alpha 7 nicotinic receptors in said neurites; and
(iii) determining whether the test compound produces a toxic effect on said neurites which is prevented by said blocker, said compound being capable of competitively binding with Synaptica Peptide to said receptors.

11 A method according to claim 9 or claim 10 wherein said neurites are GABA positive neurites present in an organotypic tissue culture of neonatal hippocampus.

12. A method for determining the ability of a compound to act as an antagonist of the polypeptide of SEQ. D No. 1 (Synaptica Peptide), which comprises determining whether said compound can inhibit the action of Synaptica Peptide or a functional analogue thereof on alpha 7 nicotinic receptors either bound to a support or presented at the surface of cells or cell membranes.

13. A method according to claim 12 wherein human SH-SY5Y neuroblastoma cells are employed or membranes derived therefrom.

14. A method for screening a compound for ability to interact with an alpha 7 nicotinic receptor, which comprises contacting said compound with said receptor, or a derivative thereof which retains a modulatory binding site for Synaptica Peptide, in the presence of Synaptica Peptide or a functional peptide variant thereof and determining whether binding of Synaptica Peptide or said variant thereof is inhibited or prevented.

15. A method as claimed in claim 14 wherein Synaptica Peptide or said variant thereof is labelled with a revealing label.

16. A method as claimed in claim 14 or claim 15 wherein said compound is found to bind to the modulatory binding site of Synaptica Peptide and its ability to act as a functional analogue or antagonist of Synaptica Peptide is determined.

17. A method of preparing a functional analogue or antagonist of Synaptica Peptide, which comprises:
(i) identifying said functional analogue or antagonist by a method according to any one of claims 1 to 13 and 16 and
(ii) synthesising said functional analogue or antagonist.

18. A method according to claim 17 wherein the compound synthesised is further incorporated into a pharmaceutical composition together with a pharmaceutically acceptable carrier or diluent.

19. A method according to claim 18 wherein said compound is an antagonist of Synaptica Peptide.

20. A functional analogue or antagonist of Synaptica Peptide identified by a method as claimed in any one of claims 1 to 13 and 16.

21. An antagonist of Synaptica Peptide as claimed in claim 20 which is capable of formulation for passage across the blood-brain barrier.

22. A pharmaceutical composition comprising a functional analogue or antagonist of Synaptica Peptide as claimed in claim 20 together with a pharmaceutically acceptable carrier or diluent.

23. A pharmaceutical composition as claimed in. claim 22 comprising an antagonist as claimed in claim 21 together with a pharmaceutically acceptable carrier or diluent.

24. Use of an antagonist of Synaptica Peptide identified in accordance with any one of claims 1 to 13 and 16 for the preparation of a medicament for treatment of a neurological disorder associated with non-enzymatic action of acetylcholinesterase.

25. A use in accordance with claim 24 wherein said neurological disorder is Alzheimer's Disease.

26. A use in accordance with claim 24 wherein said neurological disorder is Parkinson's Disease.

27. A use in accordance with claim 24 wherein said neurological disorder is Motor neuron Disease.

28. A method of treating a neurological disorder associated with non-enzymatic action of acetylcholinesterase which comprises administering an antagonist of Synaptica Peptide identified in accordance with any one of claims 1 to 13 and 16.
